(19) United States
(12) Patent Application Publication
(10) Pub. No.: US 2006/0047141 A1
(43) Pub. Date: Mar. 2, 2006

(54) Method for producing light coloured polyalkylene glycol diethyl ether of fatty acid alcanolamine

(76) Inventors: Ansgar Behler, Bottrop (DE); Frank Clasen, Hilden (DE)

Correspondence Address: COGNIS CORPORATION;PATENT DEPARTMENT, 300 BROOKSIDE AVENUE, AMBLER, PA, 19002, US

(21) Appl. No.: 10/539,737

(22) PCT Filed: Dec. 10, 2003
(86) PCT No.: PCT/EP03/13977

§ 371 Date: Jun. 20, 2005

(30) Foreign Application Priority Data
Dec. 19, 2002 (DE) 10259405.8

(51) Int. Cl. C07C 263/00

(52) U.S. Cl. 560/345

(57) ABSTRACT

The invention relates to the production of light-colored polyalkylene glycol diethyl ethers of fatty acid alkanolamines by adding ethylene to the fatty acid alkanolamide in the presence of an alkaline catalyst. The inventive method cossets of alkoxylating in the presence of reducing agents and treating the obtained product by steam in an alkaline medium.

### FIELD OF THE INVENTION

This invention relates generally to nonionic surface-active compounds and, more particularly, to a process for the production of special compounds with improved color quality and a reduced percentage content of secondary products.

### PRIOR ART

The production of alkoxylation products of fatty acid akanolamides has been known for some time and is described in detail, for example, in the overview article by Grossmann [Fette, Seifen, Anstrichmittel, 74(1), 58 (1972)]. The reaction of the alkanolamides, preferably mono-alkanolamides, with ethylene or propylene oxide is carried out in the presence of alkaline catalysts, such as tertiary amines for example [cf. EP 0557462 B1 (Berol Nobel)]. However, the disadvantage is that the reaction products are generally very discoloured and occasionally have high contents of unwanted secondary products, more particularly dioxane. Both factors limit the use of the products, more particularly for cosmetic applications.

Accordingly, the problem addressed by the present invention was to provide an improved process for the alkoxylation of fatty acid alkanolamides which would reliably avoid the disadvantages mentioned above. More particularly the products would have high color quality and a low content of unwanted secondary products, particularly dioxane.

### DESCRIPTION OF THE INVENTION

The present invention relates to a process for the production of light-colored fatty acid alkanolamide polyalkylene glycol ethers by addition of alkylene oxides onto fatty acid alkanolamides in the presence of alkaline catalysts, characterized in that the alkoxylation is carried out in the presence of reducing agents and the reaction products obtained in this way are subjected to a treatment with steam under alkaline conditions.

It has surprisingly been found that the combination of an alkoxylation in the presence of reducing agents with an after treatment with steam under alkaline conditions gives alkoxylated fatty acid alkanolamides which are both particularly light-colored and also low in unwanted secondary products. More particularly, the need for a steam treatment at high pH values was unexpected because steam treatments of water-containing surfactants are normally carried out in the neutral range. In contrast to this experience, it was found that the steam treatment at pH 6 to 7 results in a significant deterioration in color.

Fatty Acid Alkanolamides

Basically, the choice of the fatty acid alkanolamides used, which are condensation products of technical fatty acids with mono- or dialkanolamines, is not critical. The educts used are typically fatty acid alkanolamides which correspond to formula (I): 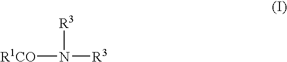
where R¹CO is a linear or branched, saturated or unsaturated acyl group containing 6 to 22 carbon atoms and 0 or 1 to 3 double bonds, R² is a hydroxyalkyl group containing 2 to 4 carbon atoms and R³ is hydrogen or has the same meaning as R². Typical examples are the condensation products of caproic acid, caprylic acid, capric acid, lauric acid, myristic acid, palmitic acid, stearic acid, isostearic acid, oleic acid, linoleic acid, linolenic acid, petroselic acid, elaeostearic acid, 12-hydroxystearic acid, ricinoleic acid, gadoleic acid, arachidonic acid, behenic acid, erucic acid and technical mixtures thereof, more particularly coconut oil fatty acid, palm kernel oil fatty acid, palm oil fatty acid and tallow fatty acid, with monoethanolamine, diethanolamine, monopropanolamine and dipropanolamine and mixtures thereof. Condensation products of coconut oil or tallow fatty acids with monoethanolamine are preferably used.
Alkylene Oxides

Suitable alkylene oxides are ethylene oxide, propylene oxide, butylene oxide or mixtures thereof. The addition may be carried out in blocks or in randomized form. The fatty acid alkanolamides and the alkylene oxides are normally used in a molar ratio of 1:1 to 1:25 and preferably 1:2 to 1:10.

Alkaline Catalysts

Besides alkali metal hydroxides and carbonates, suitable alkaline catalysts are, above all, alcoholates, more particularly sodium methylate, sodium ethylate or potassium tertbutylate. As mentioned at the beginning, tertiary amines may also be used for this purpose. The alkaline catalysts are used in quantities of typically 0.1 to 5% by weight and preferably 0.5 to 2% by weight, based on the starting materials.

Reducing Agents

Suitable reducing agents are any of the substances known by this name, for example borohydrides, more particularly sodium borohydride, and hypophosphorous acid or alkali metal salts thereof. The quantity used is generally from 0.1 to 2.5% by weight and preferably from 0.2 to 1% by weight, based on the starting materials.

Alkoxylation

The alkoxylation of the fatty acid alkanolamides may be carried out in known manner. Stirred autoclaves are generally used, being freed from adhering traces of water and atmospheric oxygen by alternate heating, evacuation and purging with nitrogen. The amides are introduced into the autoclave together with the catalyst and the reducing agent and heated to a temperature of preferably 80 to 150° C. and more preferably 110 to 140° C. The alkylene oxide is introduced in portions under a pressure of 1 to 10 and preferably 3 to 6 bar. It is advisable to follow the addition with an after-reaction time lasting one to two hours during which the temperature level can be gradually reduced. After the alkoxylation, the reaction products typically have a Gardner color value of 3 to 4.

Treatment with Steam

After cooling and expansion of the reaction mixture, the crude reaction products are subjected to a treatment with steam for which it is essential to establish an alkaline pH, preferably in the range from 9 to 12, beforehand. This is done, for example, by adding an aqueous alkali base. Steam is then passed through the mixture with continuous stirring at 100 to 120° C. until about 10 to 25% by weight of the steam used accumulates as condensate. This typically corresponds to a treatment time of ca. 60 mins. The alkoxylation product, which now has a Gardner color value of typically below 2 and a dioxane content of less than 1 ppm, is then dried.

### EXAMPLES

### Example 1

### Preparation of Coconut Fatty Acid Monoethanolamide+4EO

2,929.3 g (corresponding to 11.75 mol) of a C₈₋₁₈ coconut oil fatty acid monoethanolamide were introduced into a 5-liter stirred autoclave together with 25 g (corresponding to 0.85% by weight, based on the starting compound) of a 30% by weight methanolic solution of sodium methylate and 5.0 g of a 50% by weight aqueous solution of hypophosphoric acid (corresponding to 0.17% by weight, based on the starting compound). The autoclave was evacuated for 30 mins. at 80° C. and then purged with nitrogen. The mixture was then heated to 110° C. and 2,068.0 g (corresponding to 47 mol) ethylene oxide were introduced in portions under a pressure of up to 5 bar. The reaction time was 90 minutes. The reaction mixture was then stirred for 60 mins. at 110° C. and then for 30 mins. at 80° C. After cooling and expansion, the ethoxylated fatty acid monoethanolamide was obtained as a clear liquid (Gardner color value 3.5; hydroxyl value 168).

### Comparison Example C1

### Preparation of Coconut Oil Fatty Acid Monoethanolamide+4EO

Example 1 was repeated but without the hypophosphoric acid. The resulting ethoxylated fatty acid monoethanolamide had a Gardner color value of 3.9 and a hydroxyl value of 164.

### Example 2

### Steam Treatment of Coconut Oil Fatty Acid Monoethanolamide+4EO

1,000 g of the coconut oil fatty acid monoethanolamide+4EO prepared in accordance with Example 1 were adjusted to a pH of ca. 11 with aqueous sodium hydroxide solution and introduced into a 5-liter stirred reactor. Steam was passed through the ethoxylate while stirring at 120° C. until 20% by weight of water, based on the starting material, had condensed (which took 60 mins.). The product was then dried at 120° C./30 mbar. The end product had a Gardner color value of 1.1 and a dioxane content of less than 1 ppm.

### Example C2

### Steam Treatment of Coconut Oil Fatty Acid Monoethanolamide+4EO

1,000 g of the coconut oil fatty acid monoethanolamide+4EO prepared in accordance with Example 1 were adjusted to a neutral pH of 6.5 and introduced into a 5-liter stirred reactor. Steam was passed through the ethoxylate while stirring at 120° C. until 20% by weight of water, based on the starting material, had condensed (which took 60 mins.). The product was then dried at 120° C./30 mbar. The end product had a Gardner color value of 6.5 and a dioxane content of less than 1 ppm.

1-10. (canceled)

11. A process for the production of a light-colored fatty acid alkanolamide polyalkylene glycol ether by addition of an alkylene oxide onto a fatty acid alkanolamide in the presence of an alkaline catalyst, said process further comprising (a) carrying out the addition of alkylene oxide in the presence of a reducing agent and (b) treating the reaction product obtained in this way with steam under alkaline conditions.

12. A process according to claim 11, wherein the fatty acid alkanolamide has the formula (I): 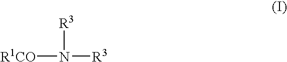
where R¹CO is a linear or branched, saturated or unsaturated acyl group containing 6 to 22 carbon atoms and 0 or 1 to 3 double bonds, R² is a hydroxyalkyl group containing 2 to 4 carbon atoms and R³ is hydrogen or has the same meaning as R².

13. A process according to claim 11, wherein the fatty acid alkanolamide is selected from a condensation product of caproic acid, caprylic acid, capric acid, lauric acid, myristic acid, palmitic acid, stearic acid, isostearic acid, oleic acid, linoleic acid, linolenic acid, petroselic acid, elaeostearic acid, 12-hydroxystearic acid, ricinoleic acid, gadoleic acid, arachidonic acid, behenic acid, erucic acid, and technical mixtures thereof, with monoethanolamine, diethanolamine, monopropanolamine and dipropanolamine and mixtures thereof.

14. A process according to claim 11, wherein the fatty acid alkanolamide is selected from a condensation product of coconut oil fatty acid, palm kernel oil fatty acid, palm oil fatty acid and tallow fatty acid with monoethanolamine, diethanolamine, monopropanolamine and dipropanolamine and mixtures thereof.

15. A process according to claim 11, wherein the fatty acid alkanolamide is selected from a condensation product of coconut oil or tallow fatty acid with monoethanolamine.

16. A process according to claim 11, wherein the alkylene oxide is selected from a group consisting of ethylene oxide, propylene oxide, butylene oxide and mixtures thereof.

17. A process according to claim 11, wherein the fatty acid alkanolamide and the alkylene oxide are used in a molar ratio of 1:1 to 1:25.

18. A process according to claim 11, wherein the alkaline catalyst is used in an amount of 0.1 to 5% by weight, based on the starting materials.

19. A process according to claim 11, wherein the reducing agent is selected from a group consisting of sodium borohydride, hypophosphorous acid and alkali metal salts thereof.

20. A process according to claim 11, wherein the reducing agent is used in an amount of 0.1 to 2.5% by weight, based on the starting materials.

21. A process according to claim 11, wherein the addition of alkylene oxide is carried out at temperatures of 80 to 150° C. and a pressure of 1 to 10 bar.

22. A process according to claim 11, wherein the treatment with steam is carried out at a pH value of 9 to 12.

23. A process for the production of a light-colored fatty acid alkanolamide polyalkylene glycol ether by addition of an alkylene oxide onto a fatty acid alkanolamide in the presence of alkaline catalysts, wherein the fatty acid alkanolamide is selected from the condensation products of coconut oil fatty acid, palm kernel oil fatty acid, palm oil fatty acid and tallow fatty acid with monoethanolamine, diethanolamine, monopropanolamine and dipropanolamine and mixtures thereof, said process further comprising (a) carrying out the addition of alkylene oxides in the presence of reducing agents and (b) treating the reaction products obtained in this way with steam under alkaline condition.

24. A process according to claim 23, wherein the fatty acid alkanolamide is selected from the condensation products of coconut oil or tallow fatty acids with monoethanolamine.

25. A process according to claim 23, wherein the alkylene oxide is selected from a group consisting of ethylene oxide, propylene oxide, butylene oxide and mixtures thereof.

26. A process according to claim 23, wherein the fatty acid alkanolamide and the alkylene oxide are used in a molar ratio of 1:1 to 1:25.

27. A process according to claim 23, wherein the alkaline catalyst is used in an amount of 0.1 to 5% by weight, based on the starting materials.

28. A process according to claim 23, wherein the reducing agent is selected from a group consisting of sodium borohydride, hypophosphorous acid or alkali metal salts thereof.

29. A process according to claim 23, wherein the reducing agent is used in amount of 0.1 to 2.5% by weight, based on the starting materials.

30. A process according to claim 23, wherein the treatment with steam is carried out at a pH value of 9 to 12.
